(19) United States
(12) Patent Application Publication
(10) Pub. No.: US 2005/0123680 A1
(43) Pub. Date: Jun. 9, 2005

(54) Micro reference electrode of implantable continous biosensor using iridium oxide, manufacturing method thereof, and implantable continuous biosensor

(76) Inventors: Sun Kil Kang, Kyonggi-do (KR); Haesik Yang, Daejon-Shi (KR); Dong Ho Shin, Daejon-Shi (KR); Hyo Kyum Kim, Daejon-Shi (KR); Youn Tae Kim, Daejon-Shi (KR)

Correspondence Address: JACOBSON HOLMAN PLLC, 400 SEVENTH STREET N.W., SUITE 600, WASHINGTON, DC, 20004, US

(21) Appl. No.: 10/861,408

(22) Filed: Jun. 7, 2004

(30) Foreign Application Priority Data
Dec. 5, 2003 (KR) 2003-88257

(51) Int. Cl. C23C 16/00; A61B 5/05; A61B 5/04; G01N 27/26

(52) U.S. Cl. 427/248.1; 600/373; 600/347

(57) ABSTRACT

Provided is a microfabricated reference electrode of an implantable continuous biosensor, a manufacturing method thereof and an implantable continuous glucose sensor using the same, providing the reference electrode of the implantable continuous biosensor comprising a metal film for an electrode formed on a dielectric substrate, and an iridium oxide film formed on the metal film for the electrode; and a manufacturing method thereof, whereby, the iridium oxide film reference electrode has a simplified manufacturing process and can employ a semiconductor batch process.

### BACKGROUND

1. Field of the Invention

The present invention relates to a microfabricated reference electrode of an implantable continuous biosensor comprising an iridium oxide film for use in a three-electrode system, such as a working electrode, a counter electrode and a reference electrode, a manufacturing method thereof and an implantable continuous glucose sensor using the same.

2. Discussion of Related Art

Recently, to manage diabetes more effectively, there is a strong demand for development of a continuous biosensor, especially glucose sensor, instead of a disposable glucose strip sensor. The continuous glucose sensor can be classified into an implantable sensor and a semi-implantable sensor according to a detection scheme.

For the implantable sensor, miniaturization, reliability and operation time are key factors, so that the development of a new microfabricated reference electrode is important to meet these requirements. Further, the glucose sensor based on an electrochemical method consists of a working electrode, a reference electrode and a counter electrode. Among them, the reference electrode remains the constant potential, serving to apply the constant potential to the working electrode. For example, although a constant voltage is applied between the working electrode and the reference electrode, if the potential of the reference electrode changes, the potential applied to the working potential will also change, thus affecting the output current.

While an Ag/AgCl reference electrode and a Calomel reference electrode in a glass tube have been widely used for a general electrochemical measurement, there is a difficulty to use them as a micro sensor due to its large volume. For the microfabricated reference electrode, the reference electrode should be placed on the same plate or wire with the working electrode and the counter electrode, if possible. Moreover, it is desirable that a method of fabricating a thin film or a thick film can be employed for forming the small reference electrode.

As a reference electrode that meets these requirements, the Ag/AgCl reference electrode fabricated using the method of fabricating the thin film or thick film can be given. The Ag/AgCl reference electrode has been widely utilized because of its large exchange current and easy formation of AgCl by chemical or electrochemical oxidization after coating the Ag film.

However, there is a problem that AgCl dissolves gradually in a water solution of a high Cl⁻ concentration. Therefore, when the dissolution of the AgCl continues to progress, the AgCl will be completely dissolved and thus only Ag will be left, resulting in a great change in the reference potential. The amount of the Ag/AgCl formed using the thin film or thick film fabrication process is a little, and moreover, the amount of the Ag/AgCl reference electrode is reduced as the Ag/AgCl becomes smaller. In this case, little amount of AgCl dissolution can influence the potential of the microfabricated Ag/AgCl reference electrode. Furthermore, a substance such as AgCl₂⁻ generated by the dissolution of the AgCl does much harm to human body, so that it is difficult to use the Ag/AgCl reference electrode inserted in or attached to the human body.

Accordingly, to solve the foregoing problems, a method for coating a relatively thick Ag film of about 10 μm and then forming the AgCl with some of it, or a method for coating a polymer film, such as Nafion, cellulose acetate and polyurethane on the AgCl, to suppress the dissolution of Ag have been employed. However, to date, the problem of AgCl dissolution is not fundamentally solved.

Karube et al. disclosed a method of fabricating a miniaturized reference electrode using an Ag/AgCl thin film in U.S. Pat. No. 6,419,809B1. This reference electrode comprises a glass substrate, a gold backbone layer, a silver layer, a dielectric thin film, a liquid junction, an electrolytic layer and a silicon passivation layer. The paper related to the above patent was released on 1998 in Sensors and Actuators B, entitled to “A novel thin-film Ag/AgCl anode structure for microfabricated Clark-type oxygen electrodes”, where the potential of the reference electrode maintains a stable potential only during 3 to 5 days due to dissolution of the AgCl. As the reference electrode area becomes smaller, the AgCl dissolution becomes very critical. For example, AgCl of the Ag/AgCl reference electrode fabricated 0.45 μm thick on 1.0×10⁻³ cm² area of microfabricated Pt electrode, dissolves out before 2 hours pass.

Meanwhile, Shin et al. developed an Ag/AgCl solid electrode using an ion selective film as a reference electrode of a potentiometric test sensor, but this was just a disposable one..

### SUMMARY OF THE INVENTION

The present invention is directed to providing a microfabricated reference electrode of an implantable continuous biosensor whose potential remains stable for a long time in the body, a manufacturing method thereof and an implantable continuous glucose sensor using the same.

To address the foregoing problems, an aspect of the present invention provides a reference electrode of an implantable continuous biosensor comprising: a metal film for an electrode formed on a dielectric substrate; and an iridium oxide film formed on the metal film for the electrode.

Meanwhile, the reference electrode of the implantable continuous biosensor can further comprise an iridium metal film formed between the metal film for the electrode and the iridium oxide film.

The dielectric substrate can be a silicon substrate on which a silicon oxide film or a silicon nitride film is formed, a glass substrate, a ceramic substrate, or a plastic substrate.

The metal film for the electrode can be made of any one of Pt, Au, C, Rh and Al.

Another aspect of the present invention provides a method of fabricating a reference electrode of an implantable continuous biosensor comprising the steps of: forming a metal film for an electrode on a dielectric substrate; and forming an iridium oxide film on the metal film.

Still another aspect of the present invention provides an implantable continuous glucose sensor comprising: an electrode film for a working electrode, an electrode film for a counter electrode and an electrode film for a reference electrode, each being formed over a dielectric substrate and separated by a dielectric film; an iridium oxide film formed on the electrode film for the reference electrode; a glucose detecting film formed on the electrode film for the working electrode; and a Teflon film and a polyurethane film coated over the overall structure.

The metal oxide film, such as an iridium oxide film, a platinum oxide film, a ruthenium oxide film, a lead oxide film, a tungsten oxide film, a titanium oxide film, an a zirconium oxide film, has a favorable potential change with respect to pH of solution, so that it can be employed as a reference electrode of the implantable continuous biosensor. Particularly, the iridium oxide layer shows good stability over a wide pH range and constant pH dependence of the potential change, so that it is desirable for a reference electrode to be used as an implantable continuous biosensor.

For a normal person, the pH of blood is in the range from 7.31 to 7.45. Thus, when analyzing elements of blood, the pH of the solution almost remains constant. In this case, a metal oxide film such as an iridium oxide film can be used as a reference electrode, since it can make a reference potential stable under a constant pH in spite of its pH dependence.

Although the pH of the solution to analyze is not constant, the analysis can be performed under the constant pH when it is used with a buffer solution having the constant pH, thus allowing the metal oxide film to be employed as a reference electrode.

Particularly, the iridium oxide film has a low current density and a potential change depending on the oxidization state of the oxide film, so that an amperometric sensor measuring a current by continuously applying a voltage is desirable for the reference electrode of the three-electrode system.

For a potentiometric method, a minute change of the reference electrode directly leads to a change of the measured voltage so that extremely stable reference electrode is required. Meanwhile, for the amperometric method, current measurement is typically performed in a biosensor or chemical sensor after applying a constant voltage.

The voltage applied at this time is high (if oxidization) or low (if reduction) enough to raise some electrochemical reaction. Here, although there are some differences between the applied voltages, the amounts of the flowing current are similar. Therefore, although there is a little change of the potential applied to the working electrode due to the potential change of the reference electrode, the amounts of the measured current are similar.

For example, for the glucose sensor that measures the glucose concentration through H₂O₂ oxidization, a voltage more than 600 mV is applied to the Ag/AgCl reference voltage in order to generate sufficient H₂O₂ oxidization at the Pt electrode. Here, for more than 600 mV, the amount of measured current is almost similar irrespective of the applied voltage. That is, for more than 600 mV, although the potential of the reference electrode is changed by as much as 100 mV, the change in the amount of the current that flows through the working electrode is not great. As a result, for the amperometric method, although there are some potential changes of the reference electrode against time, the reference electrode is still available when a voltage high (or low) enough to raise sufficient electrode reaction is applied.

Therefore, one feature of the present invention is to provide a reference electrode of an implantable continuous biosensor using the aforementioned iridium oxide film, a manufacturing method thereof and an implantable continuous glucose sensor.

### BRIEF DESCRIPTION OF THE DRAWINGS

FIG. 1 shows a plane view of an implantable continuous biosensor and a plan view of a three-electrode system according to an embodiment of the present invention.

FIG. 2A is a cross sectional view of a three-electrode system according to an embodiment of the present invention.

FIG. 2B is a cross sectional view of the three-electrode system according to another embodiment of the present invention.

FIG. 3 is a graph illustrating a potential change against time for an Ag/AgCl reference electrode according to the prior art.

FIG. 4 is a graph illustrating pH dependence of the iridium oxide film prepared according to an experimental example of the present invention by adding 0.1 M NaOH and 0.1 M HCl in a PBS solution.

FIG. 5 is a potential change graph of an iridium oxide film continuously measured for 10 days in a PBS solution for investigating potential stability of the iridium oxide film prepared according to an experimental example of the present invention.

FIG. 6 is a diagram illustrating measured potential changes of 25 iridium oxide films, prepared according to an experimental example of the present invention and kept dry in the air.

FIG. 7 is a diagram illustrating a potential change of an iridium oxide film dipped into human serum and measured against time, in order to determine whether or not the iridium oxide film according to an experimental example of the present invention can be used as a reference electrode.

FIG. 8 is a schematic diagram of an implantable continuous glucose sensor fabricated according to an embodiment of the present invention.

FIG. 9 shows calibration curves of the glucose sensor response to glucose, using an iridium oxide film reference electrode and the commercialized Ag/AgCl reference electrode in the continuous glucose sensor of FIG. 8.

FIG. 10 is a graph showing a result of animal experimentation with a continuous glucose sensor fabricated on a polyimide substrate, similar to the glucose sensor structure of FIG. 8.

### DETAILED DESCRIPTION OF PREFERRED EMBODIMENTS

The above and other features and advantages of the present invention will become more apparent to those of ordinary skill in the art from the detailed description of the preferred embodiments with reference to the attached drawings.

An embodiment of the present invention will now be described with reference to the attached drawings.

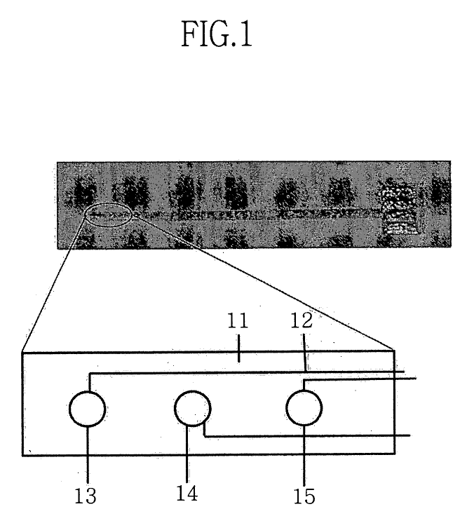

FIG. 1 shows a plane view of an implantable continuous biosensor and a plane view of a three-electrode system according to an embodiment of the present invention. An enlarged portion is a plane view of a three-electrode system comprising a reference electrode 15, a working electrode 13 and a counter electrode 14, wherein three metal electrodes 13, 14, 15 are formed on a dielectric substrate 11 and are connected to the other electrodes of the overall biosensor via a number of wirings.

The dielectric substrate 11 can be a silicon substrate on which a silicon oxide film or a silicon nitride film is formed, a glass substrate, a ceramic substrate, or a plastic substrate. The plastic substrate can be a polyimide-based polymer substrate.

The metal for forming the metal electrode can be Pt, Au, C, Rh or Al.

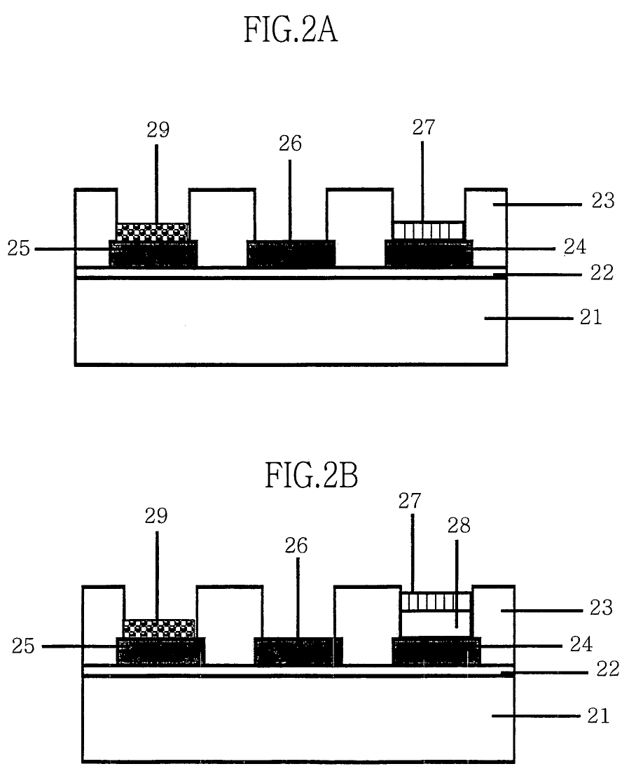

FIG. 2A is a cross sectional view of a three-electrode system according to an embodiment of the present invention, wherein, as a dielectric substrate, a silicon substrate 21 is used on which a dielectric film 22 is formed, and over which a dielectric film 23 for isolating the electrodes is formed. There exist a metal film for a working electrode 25, a metal film for a counter electrode 26 and a metal film for a reference electrode 24 among the dielectric films 23, where an iridium oxide film 27 is formed on the metal film for the reference electrode 24 and a biosensor monitoring film 29 is formed on the metal film for the working electrode 25.

The metal film for the working electrode 25, the metal film for the counter electrode 26, the metal film for the reference electrode 25 and the iridium oxide film 27 can be formed in various shapes.

Further, when the substrate is a dielectric material, such as glass, plastic or ceramic, an additional dielectric film may not be required on the substrate.

Meanwhile, FIG. 2B is a cross sectional view of the three-electrode system according to another embodiment of the present invention, and for illustration convenience, when mainly describing a difference with that of FIG. 2A, it has a structure in which an iridium metal film 28 is first formed on the metal film for the reference electrode 24, and then the iridium oxide film 27 is formed on the iridium metal film 28.

A method of fabricating the reference electrode according to an embodiment of the present invention will now be described with reference to FIG. 2A and FIG. 2B. The reference electrode is fabricated by forming a metal film for the reference electrode 24 on a dielectric substrate 21, and the iridium oxide film 27 is formed on the metal film 24. Further, the manufacturing method can further comprise the step of forming the iridium metal film 28 on the metal film for the reference electrode 24.

The iridium oxide film 27 can be formed on the metal film for the reference electrode 24 through a vacuum deposition, electrolytic deposition or thermal oxidation method, and the iridium oxide film 27 can be formed on the iridium metal film 28 through an electrochemical oxidization method.

In case of the vacuum deposition, the iridium oxide film can be formed through a reactive sputtering method that injects oxygen reactive gas into an iridium target or a direct sputtering method using an iridium oxide target.

In case of the electrolytic deposition, the iridium oxide film can be formed by preparing a solution of iridium complex, dipping this solution into the substrate where the metal film for the reference electrode 24 is formed, and applying a current or voltage to the metal film for the reference electrode 24 until the constant charges flow.

In case of the electrochemical oxidization, the iridium film can be formed by continuously alternating the electrode potentials where hydrogen and oxygen are generated in an electrolytic solution such as 0.5 M H₂SO₄.

### EXPERIMENTAL EXAMPLE 1

1,3-Phenylenediamine (MPD), hydrogen peroxide(30% solution in water), glutaraldehyde(25% solution in water), Teflon(60 wt % dispersion in water), IrCl₄, oxalic acid and K₂CO₃ were purchased from Aldrich for use in the experiment of the present invention. PBS(PH 7.4), glucose oxidase (GOₓ)(EC 1.1.3.4), glucose, poly-L-lysine hydrobromide(MW=15000-30000) were obtained from Shigma. PU(SG85A) was purchased from Thermedics Inc.(Wobum, Mass.).

This experiment was performed with a 5-inch-diameter silicon wafer. Two photomasks were used during the entire processes. After a standard cleaning procedure, a low temperature silicon oxide layer having a thickness of about 1 μm was deposited by an LPCVD method. A titanium tungsten (TiW) adhesive layer (˜750 Å) and a Pt layer(˜2000 Å) were deposited by a magnetron sputtering method. Next, a photoresist layer was coated and then exposed by a first mask. The exposed regions of Pt layer were etched by a wet etching process in 8:7:1 solution of H₂O:HCl:HNO₃. The exposed TiW was etched by an anisotropic ion etching method.

After the remaining photoresist was removed, a silicon oxide layer was deposited in a thickness of about 1 μm on the exposed platinum layer using a PECVD method and an aluminum (Al) layer (˜8000 Å) was deposited thereon. Next, the second photoresist layer was coated, and the exposed regions of the Al layer were etched by an RIE method. Then the exposed silicon oxide layer was etched by a wet etching method and the remaining photoresist layer was removed.

A Pt layer (˜2000 Å) was deposited by an e-beam evaporator for a clean Pt surface, and the remaining Al was removed by a lift-off method. The size of the exposed recessed platinum electrode is 0.1 mm².

The iridium oxide film IrOx was electrodeposited on a platinum electrode in a water solution containing 4 mM IrCl₄, 40 mM oxalic acid and 340 mM K₂CO₃. The PMPD/GOₓ(PBS) film was eletropolymerized on a microfabricated electrode at 0.7V in a PBS solution containing 5 mM MPD, 20 units/mL Gox and 1 μL/mL of 0.25% glutaradehyde. The Glucose sensor comprises a Teflon film and a PU film. The Teflon film was deposited by dipping the sensor in a 30% Teflon solution, and then by drying at room temperature for 10 minutes. This step was then repeated. The PU was deposited by dipping the sensor in a 0.4% PU solution followed by drying.

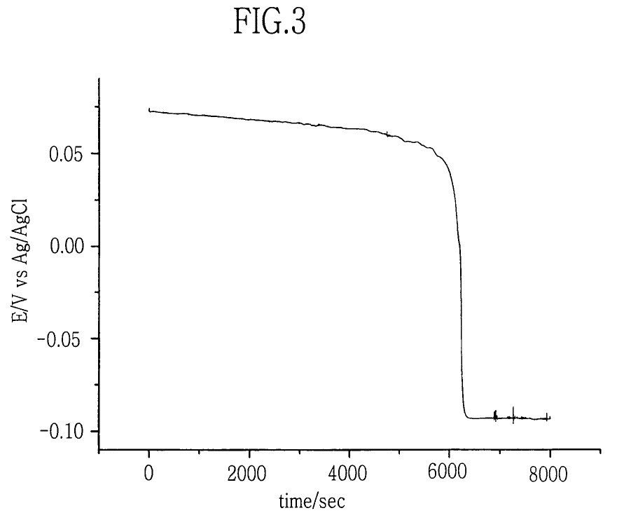

FIG. 3 is a graph illustrating a potential change against time for the commercialized Ag/AgCl reference electrode that is formed in a thickness of 0.45 82 m on 0.1 mm² Pt electrode and is dipped into a PBS buffer solution in pH 7.4. From this graph, it can be noticed that the potential of the Ag/AgCl thin film electrode is radically reduced by more than 150 mV after about 6000 seconds. This phenomenon is generated by the complete dissolution of a small amount of AgCl thin film in the PBS solution containing Cl⁻ ion. This result shows that the microfabricated Ag/AgCl reference electrode is not appropriate for the continuous glucose sensor.

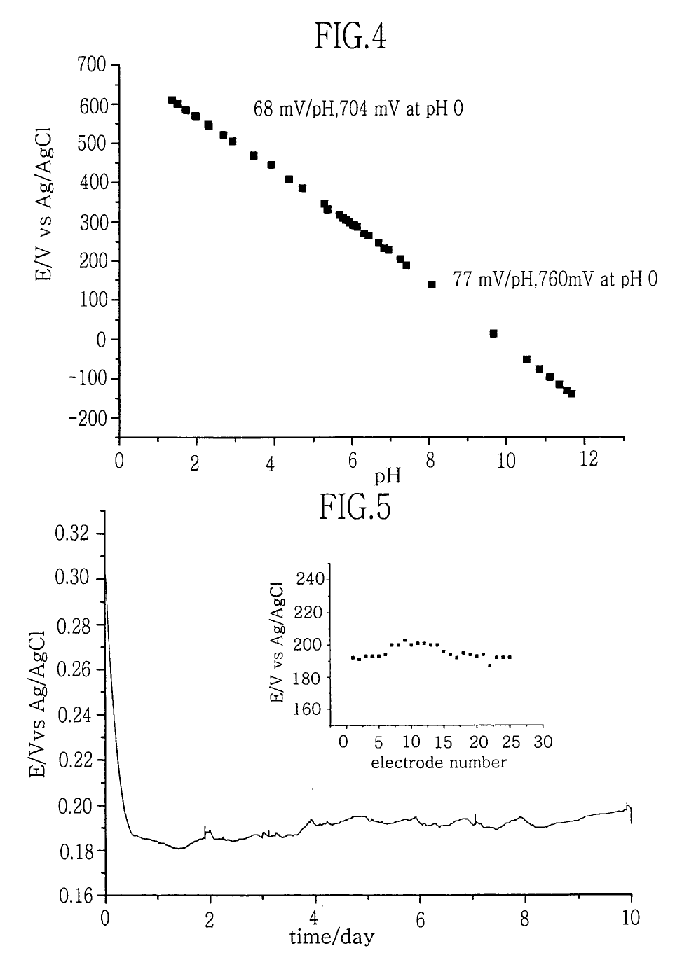

FIG. 4 is a graph illustrating pH dependence of an iridium oxide film prepared according to an experimental example of the present invention by adding 0.1 M NaOH and 0.1 M HCl in a PBS solution. A slope of the potential change has two different regions that meet near pH 6, that is, −68 mV/pH below pH 6 and −77 mV/pH above pH 6.

FIG. 5 is a potential change graph of an iridium oxide film continuously measured for 10 days in a PBS solution for investigating potential stability of the iridium oxide film prepared through the above experiment. After the stabilization for approximately 1 day, the potential is stable and its drift is less than 20 mV for next 9 days. The inset of FIG. 5 shows that the potential of the 25 iridium oxide films that are dipped into the PBS solution for 10 days has 0.195V mean value and 4 mV standard deviation.

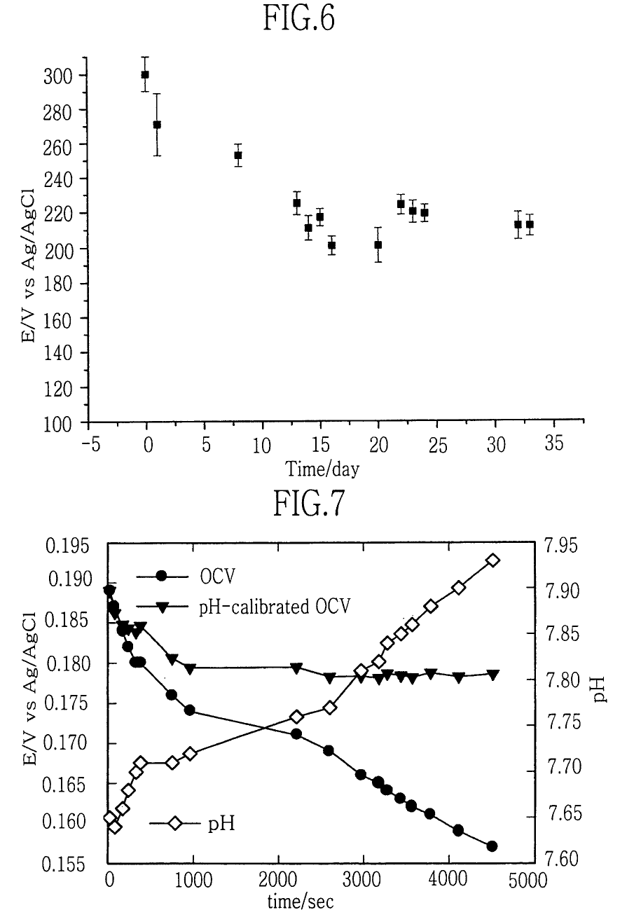

FIG. 6 is a diagram illustrating a potential change of 25 iridium oxide films, prepared through the above experiment and then measured keeping dry in the air. It shows that the 25 iridium oxide films have quite good stability and reproducibility when the potential is stabilized after 10 days.

FIG. 7 is a diagram illustrating a potential change of an iridium oxide film, which is measured against time while dipped the iridium oxide film into human serum, in order to determine whether or not the iridium oxide film fabricated in a physiological buffer solution through the above experimental can be used as a reference electrode.

The pH of serum continues to increase in open state because of the evaporation of CO₂, and the potential of the iridium oxide film is constantly changing and the pH-calibrated potential of the iridium oxide film is stable even in serum as the pH changes. Though not shown in FIG. 7, it was observed that the potential of the iridium oxide film remains stable for more than one week in serum where a bit of strong buffer solution is added to keep pH constant.

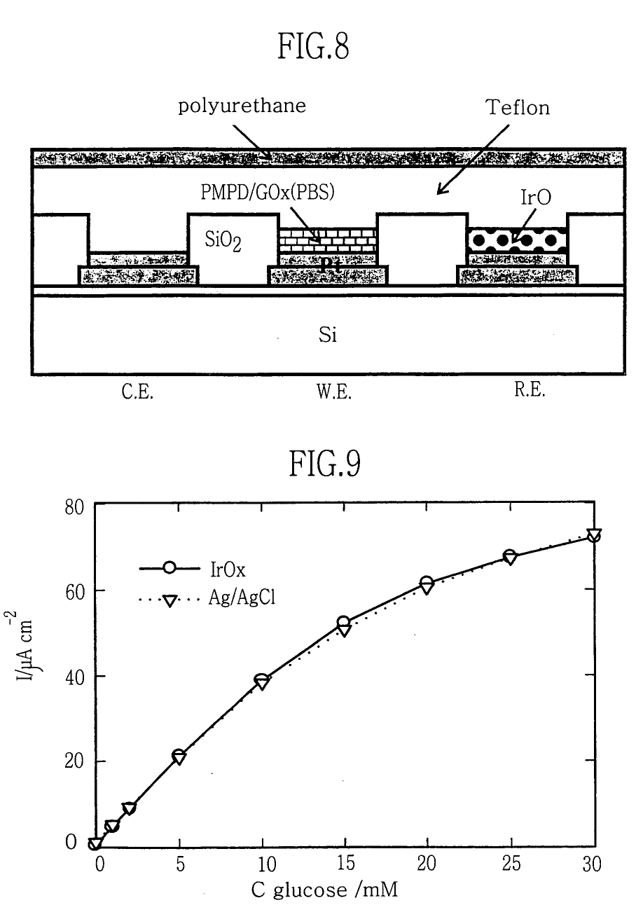

Next, a schematic diagram of an implantable continuous glucose sensor manufactured according to an embodiment of the present invention is described with reference to FIG. 8.

An array of Pt electrodes is formed on a silicon wafer over which a dielectric film is formed. Each chip comprises four electrodes and four pads. Among them, three electrodes consist of a working electrode (W.E), a counter electrode (C.E) and a reference electrode (R.E), and the iridium oxide film IrOx is formed on the reference electrode. A PMPD/GOx(PBS) thin film is formed on the working electrode as a glucose monitoring film, and the Teflon film and the polyurethane film are dip-coated. The Teflon film and polyurethane film serves to enhance stability of the reference electrode on which the iridium oxide film IrOx is formed.

FIG. 9 shows calibration curves of the glucose sensor response to glucose, using an iridium oxide film reference electrode and the commercialized Ag/AgCl reference electrode in the continuous glucose sensor of FIG. 8. Referring to FIG. 9, there is little difference of the currents flowing between the reference electrode of the iridium oxide film and the commercialized Ag/AgCl reference electrode. From this, it will be appreciated that the iridium oxide film can be operated well as a three-electrode type microfabricated reference electrode.

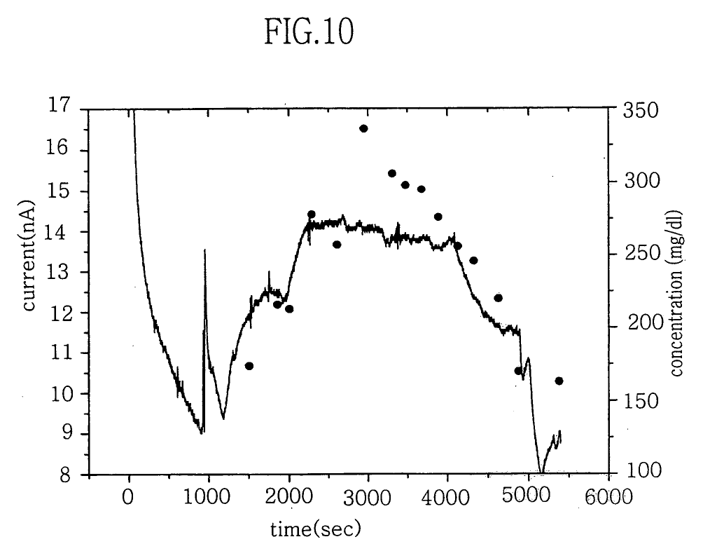

FIG. 10 is a graph showing a result of animal experimentation with a continuous glucose sensor fabricated on the polyimide substrate, similar to the glucose sensor structure of FIG. 8. The result of the animal experimentation also shows that the iridium oxide film can be operated well as a microfabricated reference electrode.

Meanwhile, other methods for fabricating the iridium oxide film are listed as follows. Experiment examples 2 and 3 illustrate a method of forming the reference electrode on the metal film through an electrolytic deposition method.

### EXPERIMENT EXAMPLE 2

A solution for the second example was prepared by dissolving 0.002-100 mM HOOCCOOH.2H₂O(oxalic acid) in 0.002-100 mM K₃IrCl₆ water solution, adding K₂CO₃ to make more than pH 9 and leaving as it is for several days. Until 10 mC/cm² to 1000 mC/cm² charges flow in this solution, the constant current was applied to Pt or Au to form the iridium oxide film.

### EXPERIMENT EXAMPLE 3

An iridium oxide film for this example was fabricated as follows. First, 75 mg IrCl₄ was put into 50 ml distilled water to melt while stirring for 30 minutes, and then 0.5 ml of 30% H₂O₂ was added and was stirred for 10 minutes. Next, 250 mg HOOCCOOH.2H₂O (oxalic acid) was added therein and was stirred again for 10 minutes, and then K₂CO₃ was added to make pH 10.5. The solution of pH 10.5 was stabilized for 2 days at room temperature. Thereafter, the iridium oxide film was formed by applying a current of 0.5-1 mA/cm2 for 6 minutes or by repeating the potential 100 times with a potential range of 0.0 V to 0.60 V with respect to the Ag/AgCl reference electrode.

Although the above description has been made with regard to the iridium oxide film, other meal oxide films such as a platinum oxide film, ruthenium oxide film, a lead oxide film, a tungsten oxide film, a titanium oxide film and a zirconium oxide film also have a favorable pH dependence of the potential change, so that they can be employed as the reference electrode of the implantable continuous biosensor.

In other words, since the above oxide films have a low current density and a potential change depending on the oxidization state of the oxide film, the three-electrode system can be employed as an implantable continuous sensor that measures a current, by continuously applying a voltage.

Although the preferred embodiments of the present invention have been described, it should be noted that these embodiments are just illustrative, and not restrictive. Further, those skilled in the art will appreciate that various modifications can be made without departing from the scope of the present invention.

As described above, in a reference electrode of the iridium oxide film, when the iridium oxide film is formed by an electrodeposition method, it can be fabricated much simpler than Ag/AgCl.

Further, it can be easily patterned using a vacuum deposition process and a lift-off process, so that a microfabricated reference electrode or a microfabricated micro array of reference electrode can be fabricated with a semiconductor batch process.

Further, the reference electrode of the iridium oxide film according to the present invention is not dissolved in the body, which maintains the constant pH, and keeps the potential stable and has bio-compatibility, so that with this reference electrode, an implantable continuous sensor that can measure a current for a long time by applying the voltage to the three-electrode system can be fabricated.

For the three-electrode system, the reference electrode of the iridium oxide film according to the present invention is still available even for the ultra-fine size, so that it can be applied to the ultra-fine sized glucose sensor system that can be continuously used inserted in or attached to the human body for a long time.

This application claims the benefit of Korean Patent Application No. 2003-88257 filed on Dec. 5, 2003, the disclosure of which is hereby incorporated herein by reference in its entirety.

1. A reference electrode of an implantable continuous biosensor comprising:
a metal film for an electrode formed on a dielectric substrate; and
an iridium oxide film formed on the metal film for the electrode.

2. The reference electrode of the implantable continuous biosensor according to claim 1, further comprising:
an iridium metal film formed between the metal film for the electrode and the iridium oxide film.

3. The reference electrode of the implantable continuous biosensor according to claim 1, wherein the dielectric substrate is any one of a silicon substrate with a dielectric film thereon, a glass substrate, a ceramic substrate, and a plastic substrate.

4. The reference electrode of the implantable continuous biosensor according to claim 3, wherein the plastic substrate is a polymer substrate of polyimide series.

5. The reference electrode of the implantable continuous biosensor according to claim 1, wherein the metal film for the electrode is made of any one of Pt, Au, C, Rh and Al.

6. A method of fabricating a reference electrode of an implantable continuous biosensor, the method comprising the steps of:
forming a metal film for an electrode on a dielectric substrate; and
forming an iridium oxide film on the metal film.

7. The method according to claim 6, further comprising the step of:
forming an iridium metal film between the step of forming the metal film for the electrode and the step of forming the iridium oxide film.

8. The method according to claim 6, wherein the step of forming the iridium oxide film on the metal film for the electrode includes the sub-steps of:
preparing a solution containing an iridium complex; and
dipping the metal film for the electrode into the solution and applying any one of a current and a voltage to the metal film until constant charges flow.

9. The method according to claim 6, wherein the step of forming the iridium oxide film on the metal film for the electrode is performed by a vacuum deposition method.

10. The method according to claim 7, wherein the iridium metal film between the metal film and the iridium oxide film is formed by the vacuum deposition method.

11. The method according to claim 7, wherein the iridium oxide film is formed by electrochemically oxidizing the iridium metal film.

12. An implantable continuous glucose sensor, comprising:
an electrode film for a working electrode, an electrode film for a counter electrode and an electrode film for a reference electrode, each being formed over a dielectric substrate and separated by a dielectric film;
an iridium oxide film formed on the electrode film for the reference electrode;
a glucose monitoring film formed on the electrode film for the working electrode; and
a Teflon film and a polyurethane film coated over the overall structure.

13. The implantable continuous glucose sensor according to claim 12, further comprising:
an iridium metal film between the metal film for the electrode and the iridium oxide film.

14. The implantable continuous glucose sensor according to claim 12, wherein the dielectric substrate is any one of a silicon substrate with a dielectric film thereon, a glass substrate, a ceramic substrate, and a plastic substrate.
